(19) United States
(12) Patent Application Publication
(10) Pub. No.: US 2019/0295691 A1
(43) Pub. Date: Sep. 26, 2019

(54) HYPERPARAMETER TUNING TO ENHANCE PREDICTIONS

(71) Applicant: Chromocare, LLC, Columbus, OH (US)

(72) Inventors: Jeff Garshon, New Albany, OH (US); Jodie Fortine, Blacklick, OH (US); James Fuller, Cary, NC (US)

(21) Appl. No.: 16/282,268

(22) Filed: Feb. 21, 2019

Related U.S. Application Data
(60) Provisional application No. 62/647,634, filed on Mar. 24, 2018.

(51) Int. Cl. G16B 40/00; G16H 20/00; G16B 20/20

(52) U.S. Cl. CPC G16B 40/00; H04L 67/42; G16B 20/20; G16H 20/00

(57) ABSTRACT

The subject disclosure relates to employing grouping and selection components to facilitate a determination of output data based on a set of scoring requirements. In an example, a method comprises retrieving, by a system operatively coupled to a processor, a set of genetic data from one or more device capable of analyzing genetic material. In another instance, the method includes identifying, by the system, a first subset of genetic data representing a star allele that corresponds to a set of phenotypic traits. In yet another aspect, the method can include generating, by the system, a set of output data based on correlations between the first subset of genetic data, clinical data and guidance data.

### CROSS REFERENCE TO RELATED APPLICATION

This application claims priority under the benefit of 35 U.S.C. § 119 to U.S. Provisional Patent Application No. 62/647,634 filed on Mar. 24, 2018, and titled “Predicting a Likelihood of Addiction”. The entirety of the disclosure of the aforementioned application is considered part of, and is hereby incorporated by reference in, the disclosure of this application.

### BACKGROUND

The practice of medicine is constantly evolving and today cutting-edge research in areas such as pharmacogenetics is impacting the way medicine is practiced. The field of pharmacogenomics refers to the study of how genes affect a body's response to medications. Despite the increased use of pharmacogenetic testing in connection with the practice of medicine, there is still much information that is not being derived from such testing. Accordingly, there is a need for technologies that address such inadequacies that currently exist.

### SUMMARY

The following presents a summary to provide a basic understanding of one or more embodiments of the invention. This summary is not intended to identify key or critical elements or delineate any scope of the particular embodiments or any scope of the claims. Its sole purpose is to present concepts in a simplified form as a prelude to the more detailed description that is presented later. In one or more embodiments described herein are systems, devices, apparatuses, computer program products and/or computer-implemented methods that facilitate an interaction between a set of agent components and execution of contracts between a subset of agent components.

According to an embodiment, a system is provided. The system comprises a processor that executes computer executable components stored in memory. The computer executable components include a transmission component configured to retrieve a set of genetic data from one or more device capable of analyzing genetic material. Further, the computer executable components include an identification component configured to identify a first subset of genetic data representing a star allele that corresponds to a set of phenotypic traits. In another aspect, the computer executable component can comprise a generation component configured to generate a set of output data based on correlations between the first subset of genetic data, clinical data and guidance data into a set of mapping data. In yet another aspect, the computer executable component can comprise a scoring component that assigns a score to respective subsets of output data based on a set of scoring requirements. Also, the computer executable components can include a determination component that determines a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

According to another embodiment, a computer-implemented method is provided. The computer-implemented method can comprise retrieving, by a system comprising a processor, a set of genetic data from one or more device capable of analyzing genetic material. The computer-implemented method can also comprise identifying, by the system, a first subset of genetic data representing a star allele that corresponds to a set of phenotypic traits. In another aspect, the computer-implemented method can comprise generating, by the system, a set of output data based on correlations between the first subset of genetic data, clinical data and guidance data into a set of mapping data. The computer-implemented method can also comprise assigning, by the system, a score to respective subsets of output data based on a set of scoring requirements. Furthermore, the computer-implemented method can comprise a determining a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

According to yet another embodiment, a computer program product for facilitating a determination of a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data. The computer program product can comprise a computer readable storage medium having program instructions embodied therewith, the program instructions executable by a processor to cause the processor to also identify a first subset of genetic data representing a star allele that corresponds to a set of phenotypic traits. The computer program product can also cause the processor to generate a set of output data based on correlations between the first subset of genetic data, clinical data and guidance data into a set of mapping data. Furthermore, the computer program product can also cause the processor to assign a score to respective subsets of output data based on a set of scoring requirements. The computer program product can also cause the processor to determine a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

### BRIEF DESCRIPTION OF THE DRAWINGS

FIG. 1 illustrates a block diagram of an example, non-limiting system that can determine a target subset of output data of the subsets of output data to present at a user interface of a device based on the target subset of output data being greater than a threshold score in accordance with one or more embodiments described herein.

FIG. 2 illustrates a block diagram of an example, non-limiting system that can summarize the set of pharmacogenetics data for presentation at a user interface.

FIG. 3 illustrates a block diagram of an example, non-limiting system that can predict a likelihood of addiction based on the risk score.

FIG. 4 illustrates a block diagram of an example, non-limiting system that can determine an impact of pharmacogenetic treatment data on the employer expenditure data.

FIG. 5 illustrates a flow diagram of an example, non-limiting computer-implemented method that facilitates a determination of a target subset of output data of the subsets of output data to present at a user interface of a device based on the target subset of output data being greater than a threshold score in accordance with one or more embodiments described herein.

FIG. 6 illustrates a flow diagram of an example, non-limiting computer-implemented method that facilitates a summarization of the set of pharmacogenetics data for presentation at a user interface in accordance with one or more embodiments described herein.

FIG. 7 illustrates a flow diagram of an example, non-limiting computer-implemented method that facilitates a prediction of a likelihood of addiction based on the risk score in accordance with one or more embodiments described herein.

FIG. 8 illustrates a flow diagram of an example, non-limiting computer-implemented method that facilitates a determination of an impact of pharmacogenetic treatment data on the employer expenditure data in accordance with one or more embodiments described herein.

FIG. 9 illustrates a block diagram of an example, non-limiting operating environment in which one or more embodiments described herein can be facilitated.

FIG. 10 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section. One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

In a non-limiting embodiment, disclosed herein are several systems employed by one or more processor that in one or more non-limiting embodiment can be integrated into a global system to form an integrated system of components executed by the one or more processor. Furthermore, any one or more features of any embodiment may be combined with any one or more other features of any other embodiment, without departing from the scope of the invention. In an instance, a first system disclosed herein is associated with pharmacological tests that provide a metabolization status for several medications (e.g., over 350 medications) amongst several therapeutic categories (e.g., anti-depressants, pain management, oncology, birth control, hematology, rheumatology, psychiatry, infectious diseases, gastroenterology, cardiovascular, endocrinology, and/or neurology).

In an aspect, the first system can facilitate the generation of actionable data representing actionable medical insights based on raw genetic data evaluated using one or more pharmacological tests. For instance, a pharmacological test can screen a panel of pharmacogenetic markers characterized as genetic variants representing gene identifiers located at different genomic positions which may cause a different response within the body (e.g., the production of varying amino acids). In an aspect, some of these important genetic variants can be referenced by use of a star allele nomenclature that creates faster and easier reference to important genes. Furthermore, the actionable data can be generated based on an identification or presence of one or more star alleles (in combination) as well as via an evaluation of clinical data corresponding to the raw genetic data. For instance, a user having multiple copies of an allele may be found to have faster metabolism of particular drugs than other users, which may require the generation of unique actionable data based on the presence of such multiple alleles and/or references in clinical data.

In one or more embodiment, the first system (e.g., system 100 disclosed below) can identify subsets of raw genetic data for use in combination with targeted guidance data to generate actionable data. A customized output (e.g., report) can be generated by appending guidance data files and integrating such data with generates actionable data representing clinically evidenced (e.g., peer reviewed) actionable insights that form a customized output for a particular patient or user given a particular circumstance. Furthermore, in a non-limiting embodiment, the guidance data files can be selected based on a mechanism (e.g., machine learning technique) that compares one or more data value score associated with the strength of supportive clinical data and the identification of one or more subset of raw genetic data to a threshold quality score (e.g., threshold data value) that represents a standard of quality that must be satisfied in order for such information to be at least a part of generated output data. In another aspect, disclosed herein is a second system capable of tracking physical genetic samples (e.g., saliva swabs, sample laboratory cups, laboratory devices) and customized unstructured as well as structured data associated with a particular client (e.g., entity, individual, etc.) and pharmacogenetic tests. Furthermore, in an aspect, a third system disclosed herein can employ algorithms that enable a determination of a client addiction susceptibility given the presence of a subset of raw genetic data associated with a group of biomarkers as well as the presence of other nuanced data (e.g., environmental factors, medical history, etc.). Furthermore, in an aspect, a fourth system disclosed herein can determine an impact of pharmacogenetics treatment data on employer expenditure data as well as efficacy of employee medical treatments.

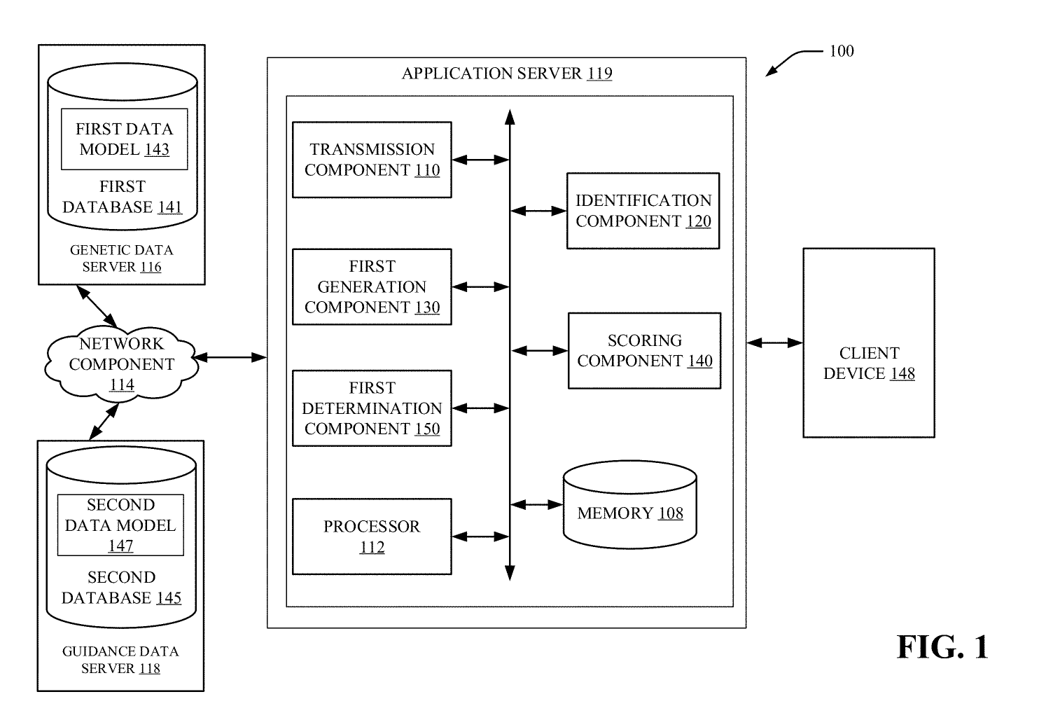

FIG. 1 illustrates a block diagram of an example, non-limiting system 100 that can facilitate a determination of a target subset of output data of the subsets of output data to present at a user interface of a device based on the target subset of output data being greater than a threshold score in accordance with one or more embodiments described herein. In an aspect, system 100 can include or otherwise be associated with one or more processor 112 that can execute the computer executable components and/or computer instructions stored in memory 108. In an aspect, system 100 can execute (e.g., using processor 112) a transmission component 110, an identification component 120, a generation component 130, a scoring component 140, and/or a determination component 150 stored in memory 108 and in a non-limiting embodiment employed on application server 119. In an aspect, one or more of the components of system 100 can be electrically and/or communicatively coupled to one or more devices of system 100 or other embodiments disclosed herein. Furthermore, system 100 can include first data model 143, first database 141, genetic data server 116, network component 114, second data model 147, second database 145, guidance data server 118, and client device 148. In an aspect, network 114 can include one or more configurable resources such as network devices network bandwidth, server devices, virtual machines, services, memory devices, storage devices, and other resources that can be provided to several user devices (e.g., one or more client device 148).

In an aspect, system 100 can execute (e.g., using processor 112 employed by application server 119) transmission component 110 to retrieve a set of genetic data from one or more device or data store capable of analyzing genetic material. For instance, a set of genetic data can comprise gene (or gene snip) information of a patient or client, biomarker data associated with a gene, star allele information, and other such information. Furthermore, transmission component 110 can retrieve such genetic data from a device (e.g., genetic data server 116) comprising one or more processor and a data store (e.g., first database 141) stored on the device. Furthermore, the genetic information can be organized in accordance with a data model (e.g., first data model 143) that can inform how one or more device (e.g., server) organizes various subsets of genetic data and defines how such subsets of data relate to other subsets of genetic data. For instance, first data model 143 can separate application information (e.g., stored at application server 119) from network information (e.g., information transmitted via network component 114). Furthermore, first database 141 can organize genetic data such as star allele data by mutation type (e.g., missense, nonsense, splice mutation, deletion, duplication, etc.) or identify a group of alleles under a single code, or provide other organizational structures to allow for efficient access of genetic data from storage devices and faster processing capabilities of the system and system components. Furthermore, a respective gene can be organized in a manner that defines a relationship between the gene, variant alleles, client customized factors (e.g., current prescribed medicines), clinical guidelines and other such relationships. Furthermore, in another aspect, the genetic information can be organized in accordance with phenotypic traits that correspond to a respective allele.

In another aspect, system 100 can also execute (e.g., via processor 112) an identification component 120 configured to identify a first subset of genetic data representing a star allele that corresponds to a set of phenotypic traits. As such, the identification component 120 can facilitate a determination of a star allele that corresponds to a set of raw genetic data stored at a data store (e.g., first database 141). In another aspect, system 100 can execute (e.g., via processor 112) a generation component 130 that can generate a set of output data based on correlations between the first subset of genetic data (and client medical history data), clinical data and guidance data. For instance, output data can represent sound information to medical practitioners implementing treatment plans for patients based on pharmacogenetics test results. Furthermore, high quality output data can be generated based on algorithms and requirements employed by generation component 130 that allow for a generation of output data that is supported by or associated with credible clinical data and guidance data by credible authorities.

For instance, the output data can include drug-drug interaction (DDI) data associated with a genetic result and a current medication a patient is using. Furthermore, the output data can include a reporting of a potential consequence of the use of a current medication in connection with a pharmacogenetic disposition associated with the user. As such, the output data can include details of a possible response (e.g., ultra-rapid metabolizer, high risk of toxicity, increased predisposition to a converting a drug into a particular byproduct, increased/decreased risk of various side effects, and other such details. Furthermore, the output data can represent informative information, actionable tasks (e.g., reduce dosage of a drug by X %), degrees of seriousness of a situation (e.g., moderate, severe, etc.), and other such information types.

However, such output data, based on DDI data for instance, can be generated based on clinical data and reports that support a finding of a DDI result represented by the output data. In another aspect, system 100 can execute (e.g., using processor 112) a scoring component 140 that can assign a score (e.g., data value) to respective subsets of output data based on a set of scoring requirements. In an aspect, scoring component 140 can assign data values to subsets of data based on scoring requirements such as whether the output data represents guidance data extracted from evidence-based guidelines, regulatory bodies, professional societies, credible authorities (e.g., CPIC, DPWG, FDA, EMA CPNDA, ACMG, etc.). Those subsets of output data without supporting evidence and not supported by credible authorities are assigned a lower score and those with supporting evidence and supported by credible authorities are assigned a higher score. As such, generation component 130 can generate actionable subsets of output data representing instructions that are suitable for implementation in a clinical setting and supported by evidence-based guidelines from credible authorities.

In another aspect, system 100 can employ a determination component 150 that can determine a target subset of output data of the subsets of output data to present at a user interface of a device (e.g., client device 148) based on a data value corresponding to the target subset of output data being greater than a threshold data value, and/or wherein the target subset of output data represents information corresponding to an absorption, metabolization, or elimination reaction of a medication in association with the first subset of genetic data. In an aspect, determination component 150 can determine, amongst several subsets of output data, a subset of output data of sufficient credibility to be transmitted to one or more client device 148 (e.g., in the form of a report) by establishing a threshold score representing a threshold quality of information to be transmitted to a client device 148. For instance, if a subset of output data is not supported by a credible authority, scoring component 140 may assign the subset of output data a score of 2 (e.g., a low score). Furthermore, in an aspect, determination component 150 may determine that a subset of output data need be assigned a score of 7.5 to qualify for transmission to a client device 148 or inclusion within a set of output communicated in another format (e.g., report). As such, system 100 can allow for the transmission of only high-quality output data to a client device 148.

In a non-limiting embodiment, transmission component 110, first generation component 130, identification component 120, first determination component 150, and scoring component 140 can be employed by an application server 119 that can represent a service layer of a technology stack of system 100. Furthermore, transmission of data and information can occur using network 114 (e.g., cloud computing network environment) representing a communication environment. Accordingly, system 100 can facilitate the generation and presentation of actionable information that can represent recommendations that are suitable for implementation in a clinical setting. For instance, phenotype data and raw genetic data can be mapped to recommendation data issued by credible third party regulatory bodies and output data can be generated and embodied in an actionable report for use by a medical physician (or other provider) to modify or proscribe a treatment plan.

In other embodiments, subsets of output data can also include informative data, moderate data, and/or serious data. In another non-limiting embodiment, system 100 can retrieve raw genetic data directly from sensor devices employed by instruments used to analyze the raw genetic data and such data can be retrieved directly from such instruments by transmission component 110. In another aspect, output data generated by system 100 can eliminate from a listing, respective drugs for use in treatment that have no supporting clinical data or non-authoritative supporting data to show that a pharmacogenetic variant found within a patient is correlated to such drug. As such, data that could be damaging to an individual is not transmitted as output data to a client device. Furthermore, data with higher confidence intervals of efficacy are transmitted as output data.

In a non-limiting embodiment, the output data can include summaries of drugs, drug classes, pharmacogenetic results related to a users' use of such drugs, and/or a listing of drugs that interact with the drug. In yet another aspect, the output data can be generated to include risk management data to indicate a risk level associated with the potential occurrence of particular medical conditions based on the pharmacogenetic results of each user. For instance, a subset of output data can indicate that a client has a moderate risk of antipsychotic induced weight gain due the presence of a Taq1A gene variant for an individual. In another aspect, a subset of output data can represent detailed guidance data associated with a drug and such output data can also include actionable insights as well. In yet another aspect, a subsets of output data can include details associated with pharmacogenetic testing such as a gene, genotype, phenotype, and alleles tested. In another aspect, other subsets of output data can represent a list of inhibitors and a patient information card.

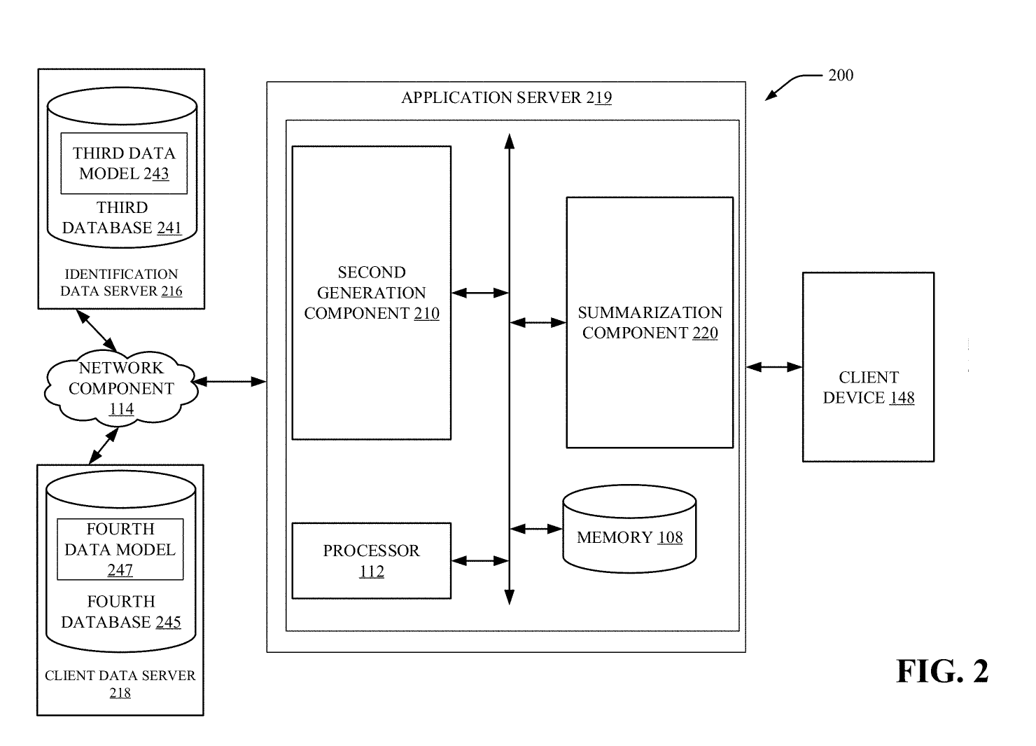

Turning now to FIG. 2, illustrated is a block diagram of an example, non-limiting system 200 that can summarize the set of pharmacogenetics data for presentation at a user interface. Repetitive description of like elements employed in other embodiments described herein is omitted for sake of brevity. In an aspect, system 200 can include a second generation component 210, a summarization component 220, processor 112, memory 108, application server 219, client device 148, identification data server 216, client data server 218, and network component 114. In an aspect, system 200 can execute (e.g., using processor 112) a second generation component 210 configured to generate a set of pharmacogenetics data based on a coupling of a set of identification data to a set of client data. Furthermore, system 200 can also employ a summarization component 220 configured to summarize the set of pharmacogenetics data for presentation at a user interface of a client device.

In an aspect, system 200 can execute (e.g., using a processor 112) a second generation component 210 configured to generate a set of pharmacogenetics data based on a coupling of a set of identification data to a set of client data. In an aspect, the set of pharmacogenetic data can include laboratory data, demographic information of a patient, provider information, medication a patient is taking, diagnostic codes, medications a provider is authorized to distribute, star alleles associated with patient genetic information, requisition form information, laboratory testing status information, and other such information. In an aspect, system 200 can execute (e.g., via processor 112) a second generation component 210 that can generate all such pharmacogenetic data and couple such data to identification data. In an aspect, the identification data can represent a unique identifier associated with a patient, laboratory, provider, or other such entity. Accordingly, second generation component 210 can not only generate data but also organize a range of data sets associated with an entity and classify such information for easier searching, tracking, and monitoring over a period of time.

In a non-limiting embodiment, system 200 can include an integration component that integrates system 200 components to a laboratory information system in order to facilitate a secure transfer of data between a laboratory entity and an internal system that can be accessed by client devices (e.g., patient portal, organization/enterprise portal, broker portal). Furthermore, in an aspect, the integration component of system 200 can allow for the intake of data points representing laboratory requisition information, shipping tracking information of patient samples, and other such information. Furthermore, in a non-limiting embodiment, system 200 can employ summarization component 220 that can summarize the set of pharmacogenetics data for presentation at a user interface (e.g., client device display). For instance, summarization component 210 can present a summary of the pharmacologic data (e.g., via a dashboard) to provide a user with access to generated pharmacogenetic information associated with an identifier.

Furthermore, the output data generated by system 100 can also be accessed via system 200 and summarized using summarization component 210. Accordingly, system 100 and system 200 can be integrated into a holistic system in a non-limiting embodiment. In another aspect, system 200 can facilitate the tracking of raw genetic data associated with laboratory instruments via sensor devices employed by such laboratory instruments. For instruments, a laboratory sample of saliva being processed can provide data as to the status of the processing or status of the sample based on sensor employed by laboratory storage cups (e.g., that contain the saliva) or laboratory processing equipment that analyze the sample and extract raw genetic data. In yet another non-limiting embodiment, second generation component 210 can generate the identification data based on structured or unstructured identification data accessed from identification data server 216 which is stored in third database 241 in accordance with an organizational framework (e.g., third data model 243). Also, system 200 can execute (e.g., using processor 112) second generation component 210 to access client data from client data server 218 which can be configured to store client data within a fourth database 245 that is organized in accordance with a fourth data model 247.

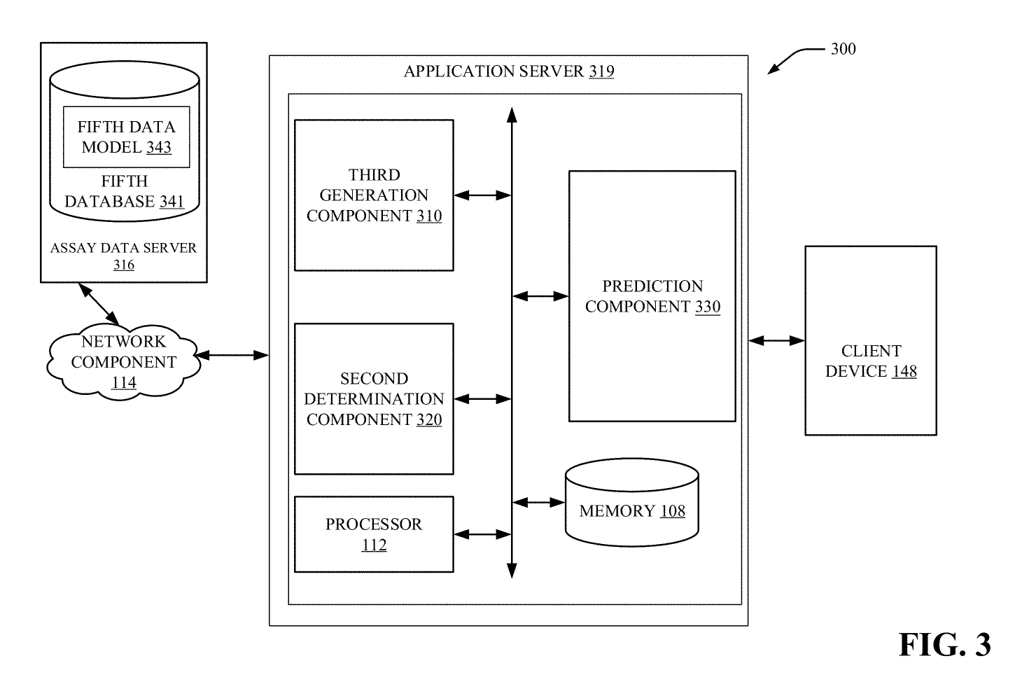

Turning now to FIG. 3, illustrated is a block diagram of an example, non-limiting system 300 that can predict a likelihood of addiction based on the risk score. Repetitive description of like elements employed in other embodiments described herein is omitted for sake of brevity. In an aspect, system 300 can execute (e.g., using processor 112) a third generation component 310, second determination component 320, and/or prediction component 330. In an aspect, system 300 can execute (e.g., using processor 112) a generation component 310 configured to generate assay data corresponding to a group of biomarkers representing pharmacogenetic factors that indicate addiction susceptibility. For instance, generation component 310 can generate assay data representing a group of biomarkers (e.g., 75) that have been shown in clinical studies to influence a likelihood that an individual may become addicted to an item. In an aspect, generation component 310 can access assay data server 316 that includes structured and/or unstructured assay data and can be configured as a fifth database 341 in accordance with a fifth data model 343 organizational framework. Furthermore, in an aspect, system 300 can execute a determination component 320 that can determine a risk score based on the generated assay data based on a set of weighting factors. For instance, determination component 320 can employ an algorithm that weights the importance of individual biomarkers in association with a risk level of addiction given the presence of a certain set of pharmacogenetic factors. In essence, determination component 320 represents a scoring mechanism for establishing a proclivity for addiction for a patient with a particular set of factors.

In an aspect, determination component 320 can analyze and identify a unique set of biomarkers outside of the scope of traditional pharmacogenetic testing that indicate addiction susceptibility. For instance, the assay data can represent panel for testing that evaluates the effect of opiates on a patient based on how the patients' body (e.g., as indicated by metabolization genes) metabolizes opiates. Furthermore, such addiction markers (e.g., represented by assay data) can include physical markers, neurological markers, psychological (e.g., behavioral) markers, and other such markers to determine the individuals' proclivity for addiction. As a non-limiting example, generation component 310 can generate assay data based on a review of duplicate genes associated with reward center markers in an individual. For instance, an individual with two or three copies of a DRD star allele may be determined (e.g., by determination component 320) to have a higher likelihood of compulsivity, risk taking, or documented behavioral habits that indicate a higher risk of becoming addictive to opiates.

In another instance, neuro-receptors in a users' brain can be sites for opiate binding and individuals with more of such receptor sites (as determined by assay date generated by generation component 310) may lead to greater euphoric effects felt by such individual after intaking opiates, which may also indicate a greater susceptibility to opiate addiction. As such, determination component 320 can employ an algorithm that utilizes the weight of each biomarker analyzed in a tested panel in order to create an addiction risk score. The addiction risk score can represent a data value that represents an uncalibrated metric of addiction susceptibility of an individual with respect to a target item (e.g., opiate). In an aspect, the weighting factors can include a number of clinical studies tied to a target biomarker. For instance, a weighting factor can include the detection of a target markers presence can indicate a susceptibility to opioid addiction, nicotine or other such potentially addictive item and such. Other weight factors can include the presence of neuro-receptors (e.g., quantity of receptors present in an individual), the presence of star alleles (e.g., number of copies of such star alleles), demographic information, environmental information, and other such information. Furthermore, each respective weighting factor can be assigned s risk score (e.g., using second determination component 320) and such risk score.

In another aspect, system 300 can employ a prediction component 330 that can predict a likelihood of addiction based on the risk score. As such, prediction component 330 can employ an algorithm that calibrates the risk score using data comparison techniques and pattern recognition to provide context of the risk score in light of larger population data. Accordingly, prediction component 330 can predict whether an individual given a set of factors has a low, moderate, or high risk of susceptibility to addiction (with respect to a potentially addictive item). In a non-limiting embodiment, prediction component 330 can employ a machine learning algorithm to compare the risk score and/or assay data to training data to adjust a risk score based on a set of data that presented particular outcomes in larger population data sets.

In an aspect, prediction component 330 can utilize machine learning approaches to interpret meaningful relationships between phenotypic data and genotypic data, biomarker relationships, genetic data and disease presentations (e.g., asthma, chronic diseases, mental disorders, etc.), linkages between genetic variations and medication efficacy. In an aspect, prediction component 330 can employ machine learning methods to build a relationship model to determine addiction susceptibility given a set of assay data, evaluate and modify the machine learning model (automatically) in light of updated data and feedback data, as well as allow the model to make predictions and adjust predictions based on outcome data. In non-limiting embodiments, prediction component 330 can utilize machine learning techniques including model-based integration approaches, probabilistic causal network frameworks, ensemble classifier frameworks, concatenation-based integration approaches, transformation-based integration approaches, and/or data reduction and feature selection approaches to predict addiction susceptibility based on data sets (e.g., assay data, pharmacogenetic data, environmental data, epigenetic data, etc.). Thus, overtime prediction component 330 can gain more insight into collected samples and calibrate risks more finely. Furthermore, scoring systems of system 300 can become more robust and system 300 can provide guidance as to forms of recovery treatment that are available to users (e.g., based on a genetic profile of the user).

In another non-limiting embodiment, system 300 can employ prediction component 330 to adjust scoring and weighting techniques based on new data as well as identify new patterns associated with such data. For instance, as hundreds of thousands of patients' data is collected, a biomarker can better be determined to be associated with a low weight or high weight that indicates that a number of patient having a condition has a particular level of susceptibility to addiction. Furthermore, system 300 can intake data representing new genetic snips and determine whether such snip data is more or less impactful in determining addiction susceptibility over time. For instance, system 300 can suggest new biomarkers to be added to a testing panel, identify target areas that need to be added to core products and assays. Furthermore, system 300 can employ artificial-intelligence techniques to find new biomarkers that may be relevant to a testing panel. As such, panels can be determined by system 300 to identify addiction susceptibility to opioid, nicotine, and alcohol based on opioid biomarkers, nicotine biomarkers, and alcohol biomarkers respectively. Furthermore, prediction component 330 can identify trends between biomarkers such as a high susceptibility to alcohol addiction correlates to high susceptibility to nicotine addiction based on pharmacogenetic data associated with a sample population.

In another aspect, prediction component 330 can employ machine learning models that incorporate learnings from previous analysis of trends and techniques. As such, prediction component 330 can tune hyperparameter values in a model to appropriately fit the problem or predictive solution sought. Furthermore, prediction component 330 can employ cross validation (e.g., k-fold cross validation), back testing and/or regularization techniques to optimize the machine learning models for use to determine addiction susceptibility of a target user. As such, these techniques can ensure that metrics used for optimization correlate well to unseen data. In an aspect, addiction susceptibility data can be analyzed by a machine learning model employed by prediction component 330 and features can be extracted from such data to observe how accurate a prediction for a likelihood of addiction susceptibility turned out. Furthermore, in an aspect prediction component 330 can employ optimization techniques to suggest tuning to the hyper-parameters and again employ the machine learning model with tuned hyper-parameters on the raw data to determine the increase in predictive accuracy from the tuning.

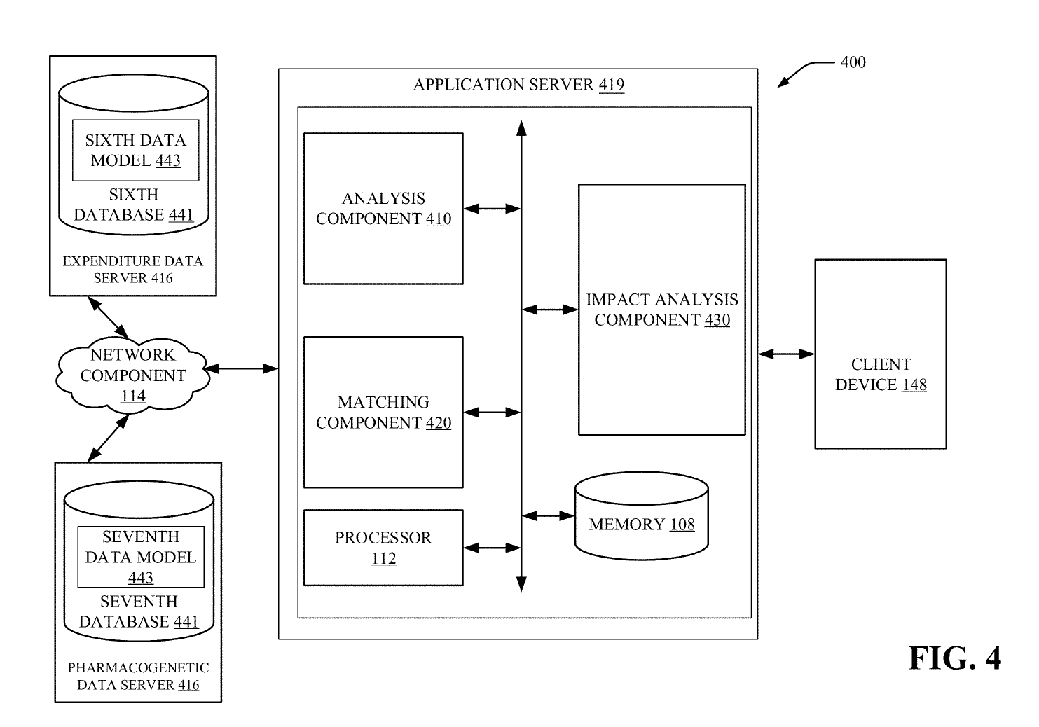

Turning now to FIG. 4, illustrated is a block diagram of an example, non-limiting system that can determine an impact of pharmacogenetic treatment data on the employer expenditure data. Repetitive description of like elements employed in other embodiments described herein is omitted for sake of brevity. In an aspect, system 400 can employ an analysis component 410, matching component 420, and/or impact analysis component 430. In an aspect, system 400 can represent a tool that facilitates the analysis of medical claim data, emergency room data, hospitalization data, surgery data, prescription claim data, and/or other such data sets associated with an client base (e.g., employees insured by a self-insuring organization). In an aspect, system 400 can employ analysis component 410 to evaluate a set of employer expenditure data. For instance, analysis component 410 can analyze pharmaceutical data and medical spend data for an employer. Furthermore, analysis component 410, using healthcare trend data and insurance expenditure data, can determine individuals covered under an insurance plan that could benefit (e.g., lower insurance costs of employer) from pharmacogenetic testing. In an aspect, analysis component 410 can utilize machine learning and artificial intelligence techniques to identify high expense inducing activities as supported by data (DDI data, star alleles within an employee population group and/or identification of class of medications that exacerbates susceptibility to undergoing a condition. Furthermore, analysis component 410 can facilitate a recommendation of the prescription of drugs that would need pharmacogenetic guidance.

In another aspect, system 400 can employ a matching component 420 that can match the employer expenditure data to a set of pharmacogenetic data. For instance, by matching such data matching component 420 can pair pharmacogenetic data representing genetic variations with recommended medications to be taken by individuals with such genetic variations, common DDI interaction data associated with such variations, and medications prescribed to the individual with such genetic variation. Furthermore, system 400 can employ an impact analysis component 430 that can determine an impact of pharmacogenetic treatment data on the employer expenditure data. For instance, impact analysis component 430 can determine that a known side-effect of a medication taken by an individual is occurring in such individual and causing an increase in insurance costs to the employer.

Furthermore, the administration of a pharmacogenetic test may indicate the presence of unfavorable DDI with such individual and could result in a determination to switch medications for the user based on such pharmacogenetic testing. As such impact analysis component 430 can determine the potential impact (E.g., cost savings) to an employer that administering pharmacogenetic testing to particular employees can have on the employee's well-being and on health costs to the organization. For instance, the result of pharmacogenetic testing to particularly identified patients can indicate a need for medication change, and medication dosage change that can result in fewer hospitalizations of such employee and a lower healthcare expense payout. Furthermore, analysis component 410 can analyze hospitalization data, frequency of medication change data, dosage change data, emergency room visit data, and other such data over a period of time to determine whether such person is a candidate for pharmacogenetic testing. Furthermore, system 400 can also integrate with system 200 to track insurance spend data, medical claim data, pharmacy claim data and post-testing result data to determine an impact of pharmacogenetic testing on employer healthcare costs. In a non-limiting embodiment, any combination of system 100, system 200, system 300, and/or system 400 can be integrated together and executed in combination to solve any of the issues addressed in this disclosure.

Aspects disclosed herein can be integrated with the tangible and physical infrastructure components of one or more oil and gas exploration equipment at one or more localities. In another aspect the systems and methods disclosed can be integrated with physical devices such as sucker-rod pumping devices, tablets, desktop computers, mobile devices, and other such hardware. Furthermore, the ability to employ iterative machine learning techniques to analyze and identify trends associated with pharmacogenetic data associated with cannot be performed by a human. For example, a human is unable to group pharmacogenetic data from several sources and covering a large range of biomarkers simultaneously based on machine learning and artificial intelligence comparative techniques in an efficient and accurate manner. Thus, the systems, methods, and computer program products disclosed herein solve new and unique problems that did not previously exist. In an aspect, the disclosed subject matter allows for the facilitation of a relationship between mechanical equipment components of laboratory equipment device technology and computer-implemented components that identify tracking data and pharmacogenetic data in mechanical laboratory equipment devices.

For simplicity of explanation, the computer-implemented methodologies are depicted and described as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts can be required to implement the computer-implemented methodologies in accordance with the disclosed subject matter. In addition, those skilled in the art can understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

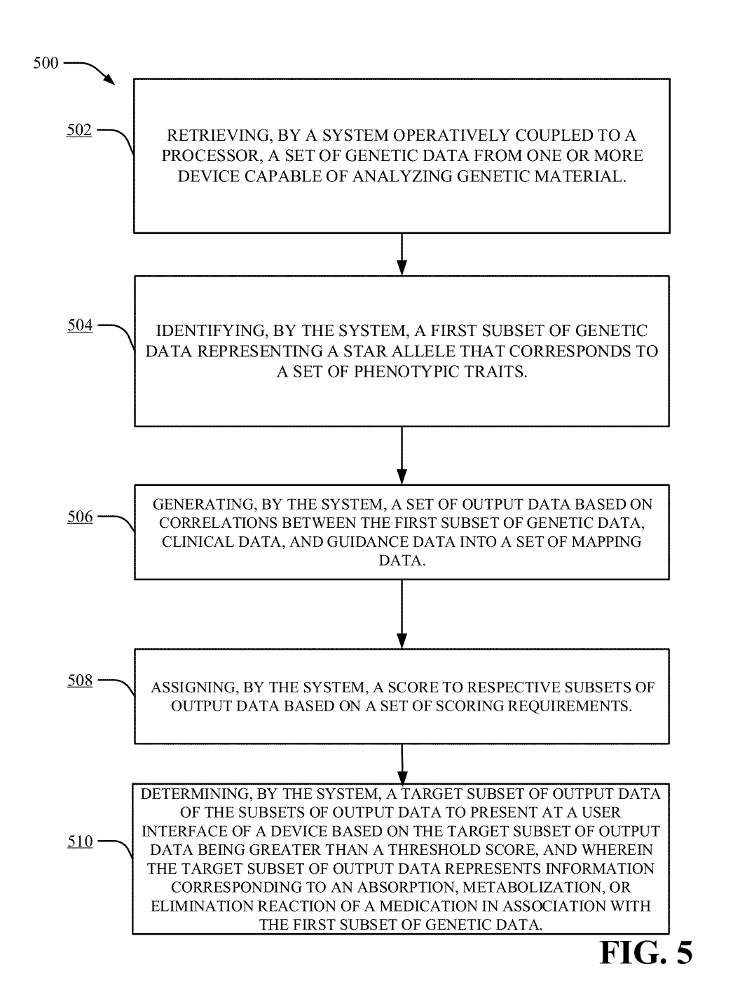

FIG. 5 illustrates a flow diagram of an example, non-limiting computer-implemented method 500 that facilitates a determination of a target subset of output data of the subsets of output data to present at a user interface of a device based on the target subset of output data being greater than a threshold score in accordance with one or more embodiments described herein. In an aspect, one or more of the components described in computer-implemented method 500 can be electrically and/or communicatively coupled to one or more devices. Repetitive description of like elements employed in other embodiments described herein is omitted for sake of brevity. In some implementations, at reference numeral 502, a set of genetic data can be retrieved (e.g., using transmission component 110) from one or more device capable of analyzing genetic material. At 504, a first subset of genetic data representing a star allele that corresponds to a set of phenotypic traits can be identified (e.g., using identification component 120). At 506, a set of output data can be generated (e.g., using first generation component 130) based on correlations between the first subset of genetic data, clinical data and guidance data. At 508, a score can be assigned (e.g., using scoring component 140) to respective subsets of output data based on a set of scoring requirements. At 510, a target subset of output data can be determined (e.g., using first determination component 150) of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

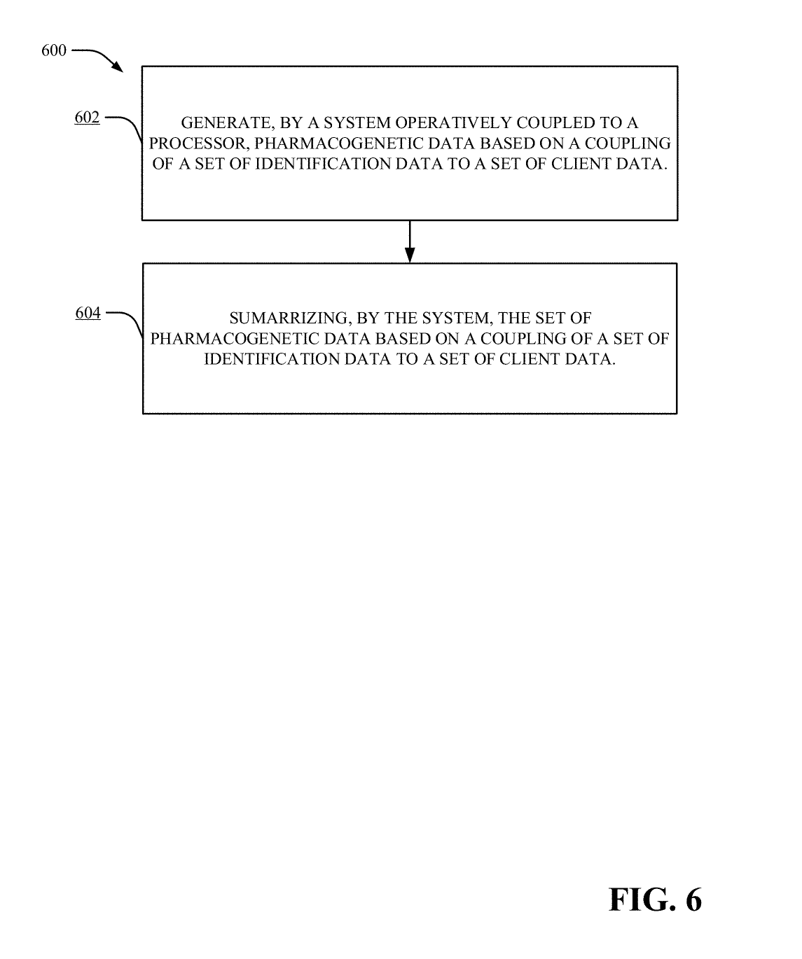

FIG. 6 illustrates a flow diagram of an example, non-limiting computer-implemented method 600 that facilitates a summarization of the set of pharmacogenetics data for presentation at a user interface in accordance with one or more embodiments described herein. In an aspect, one or more of the components described in computer-implemented method 600 can be electrically and/or communicatively coupled to one or more devices. Repetitive description of like elements employed in other embodiments described herein is omitted for sake of brevity. In some implementations, at reference numeral 602, pharmacogenetics data is generated (e.g., using second generation component 210) based on a coupling of a set of identification data to a set of client data. At reference numeral 604, the set of pharmacogenetics data is summarized (e.g., using summarization component 220) for presentation at a user interface.

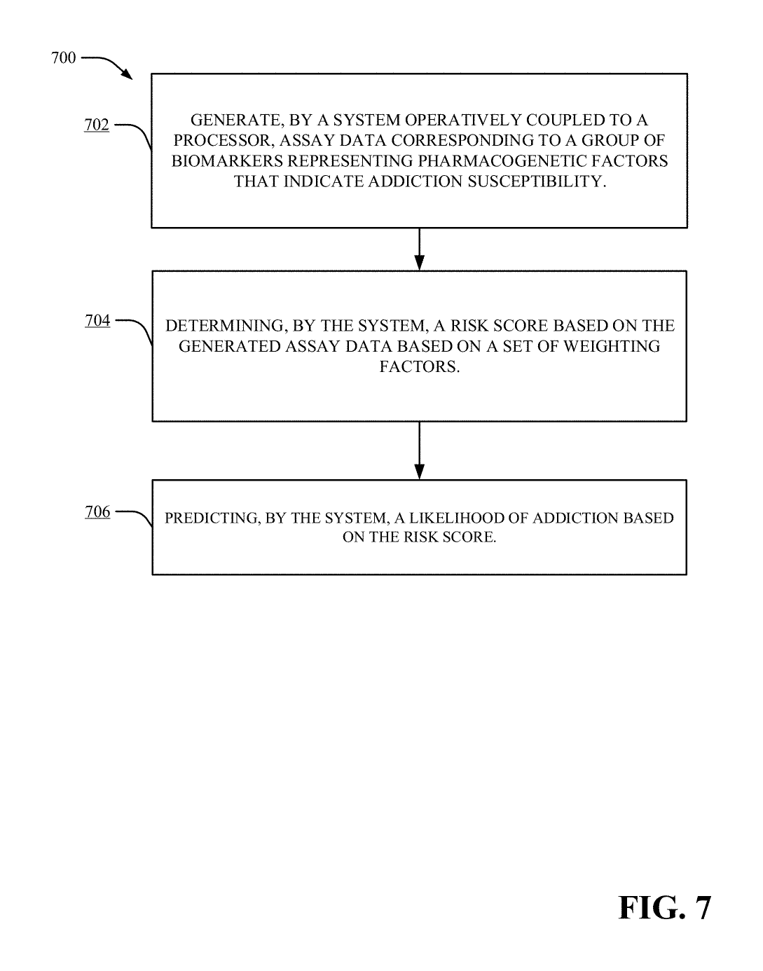

FIG. 7 illustrates a flow diagram of an example, non-limiting computer-implemented method that facilitates a prediction of a likelihood of addiction based on the risk score in accordance with one or more embodiments described herein. In an aspect, one or more of the components described in computer-implemented method 700 can be electrically and/or communicatively coupled to one or more devices. Repetitive description of like elements employed in other embodiments described herein is omitted for sake of brevity. In some implementations, at reference numeral 702, assay data corresponding to a group of biomarkers representing pharmacogenetic factors is generated (e.g., using third generation component 310) that indicate addiction susceptibility. At reference numeral 704, a risk score is determined (e.g., using second determination component 320) based on the generated assay data based on a set of weighting factors. At reference numeral 706, a likelihood of addiction is predicted (e.g., using prediction component 330) based on the risk score.

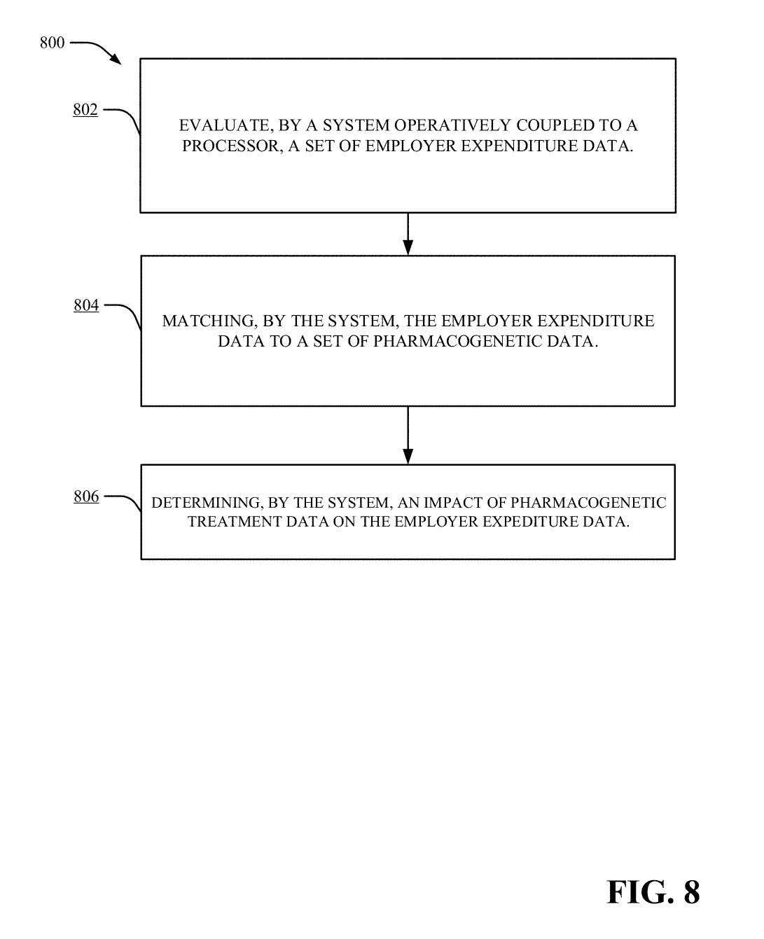

FIG. 8 illustrates a flow diagram of an example, non-limiting computer-implemented method that facilitates a determination of an impact of pharmacogenetic treatment data on the employer expenditure data in accordance with one or more embodiments described herein. In an aspect, one or more of the components described in computer-implemented method 800 can be electrically and/or communicatively coupled to one or more devices. Repetitive description of like elements employed in other embodiments described herein is omitted for sake of brevity. In some implementations, at reference numeral 802, a set of employer expenditure data is evaluated (e.g., using analysis component 410). At reference numeral 804, the employer expenditure data is matched (e.g., using matching component 420) to a set of pharmacogenetic data. At reference numeral 806, an impact of pharmacogenetic treatment data on the employer expenditure data is determined (e.g., using impact analysis component 430).

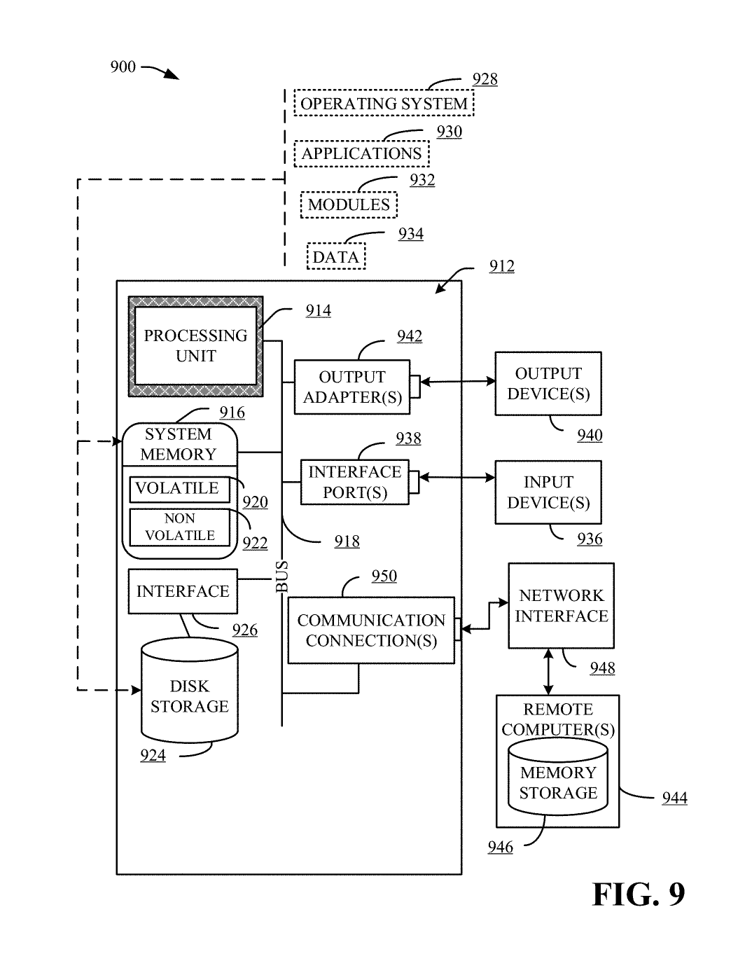

FIG. 9 illustrates a block diagram of an example, non-limiting operating environment in which one or more embodiments described herein can be facilitated. In order to provide a context for the various aspects of the disclosed subject matter, FIG. 9 as well as the following discussion is intended to provide a general description of a suitable environment in which the various aspects of the disclosed subject matter can be implemented. FIG. 9 illustrates a block diagram of an example, non-limiting operating environment in which one or more embodiments described herein can be facilitated. With reference to FIG. 9, a suitable operating environment 900 for implementing various aspects of this disclosure can also include a computer 912. The computer 912 can also include a processing unit 914, a system memory 916, and a system bus 918. The system bus 918 couple's system components including, but not limited to, the system memory 916 to the processing unit 914. The processing unit 914 can be any of various available processors. Dual microprocessors and other multiprocessor architectures also can be employed as the processing unit 914. The system bus 918 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 916 can also include volatile memory 920 and nonvolatile memory 922. The basic input/output system (BIOS), containing the basic routines to transfer information between elements within the computer 912, such as during start-up, is stored in nonvolatile memory 922. By way of illustration, and not limitation, nonvolatile memory 922 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 920 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 912 can also include removable/non-removable, volatile/non-volatile computer storage media. FIG. 9 illustrates, for example, a disk storage 924. Disk storage 924 can also include, but is not limited to, devices like a magnetic disk drive, floppy disk drive, tape drive, Jaz drive, Zip drive, LS-100 drive, flash memory card, or memory stick. The disk storage 924 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 924 to the system bus 918, a removable or non-removable interface is typically used, such as interface 926. FIG. 9 also depicts software that acts as an intermediary between users and the basic computer resources described in the suitable operating environment 900. Such software can also include, for example, an operating system 928. Operating system 928, which can be stored on disk storage 924, acts to control and allocate resources of the computer 912.

System applications 930 take advantage of the management of resources by operating system 928 through program modules 932 and program data 934, e.g., stored either in system memory 916 or on disk storage 924. It is to be appreciated that this disclosure can be implemented with various operating systems or combinations of operating systems. A user enters commands or information into the computer 912 through input device(s) 936. Input devices 936 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 914 through the system bus 918 via interface port(s) 938. Interface port(s) 938 include, for example, a serial port, a parallel port, a game port, and a universal serial bus (USB). Output device(s) 940 use some of the same type of ports as input device(s) 936. Thus, for example, a USB port can be used to provide input to computer 912, and to output information from computer 912 to an output device 940. Output adapter 1242 is provided to illustrate that there are some output device 940 like monitors, speakers, and printers, among other such output device 940, which require special adapters. The output adapters 942 include, by way of illustration and not limitation, video and sound cards that provide a means of connection between the output device 940 and the system bus 918. It should be noted that other devices and/or systems of devices provide both input and output capabilities such as remote computer(s) 944.

Computer 912 can operate in a networked environment using logical connections to one or more remote computers, such as remote computer(s) 944. The remote computer(s) 944 can be a computer, a server, a router, a network PC, a workstation, a microprocessor based appliance, a peer device or other common network node and the like, and typically can also include many or all of the elements described relative to computer 912. For purposes of brevity, only a memory storage device 946 is illustrated with remote computer(s) 944. Remote computer(s) 944 is logically connected to computer 912 through a network interface 948 and then physically connected via communication connection 950. Network interface 948 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 950 refers to the hardware/software employed to connect the network interface 948 to the system bus 918. While communication connection 950 is shown for illustrative clarity inside computer 912, it can also be external to computer 912. The hardware/software for connection to the network interface 948 can also include, for exemplary purposes only, internal and external technologies such as, modems including regular telephone grade modems, cable modems and DSL modems, ISDN adapters, and Ethernet cards.

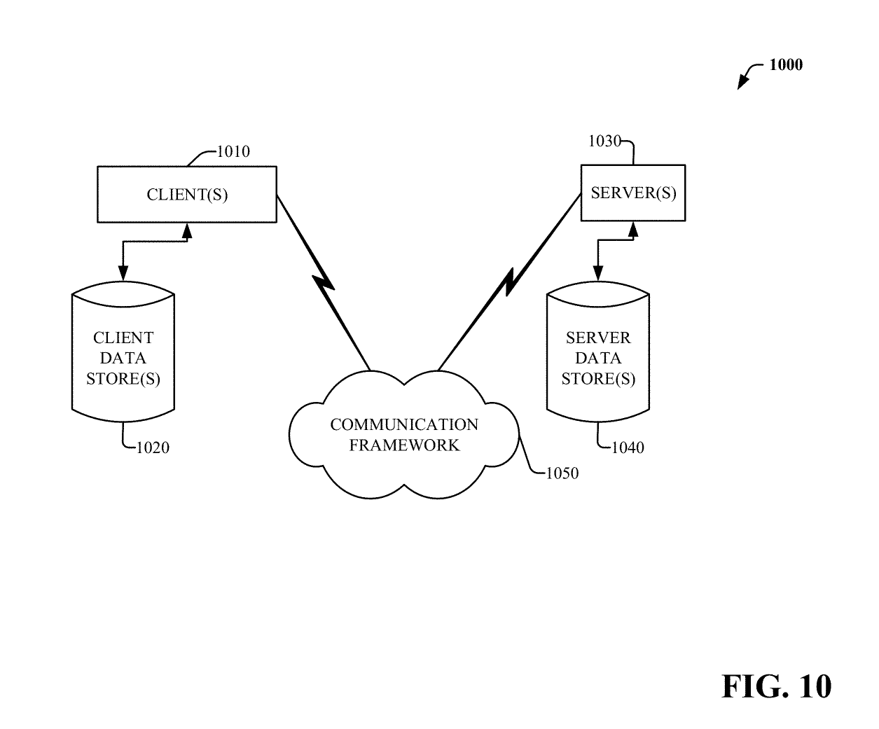

Referring now to FIG. 10, there is illustrated a schematic block diagram of a computing environment 1000 in accordance with this disclosure. The system 1000 includes one or more client(s) 1002 (e.g., laptops, smart phones, PDAs, media players, computers, portable electronic devices, tablets, and the like). The client(s) 1002 can be hardware and/or software (e.g., threads, processes, computing devices). The system 1000 also includes one or more server(s) 1004. The server(s) 1004 can also be hardware or hardware in combination with software (e.g., threads, processes, computing devices). The servers 1004 can house threads to perform transformations by employing aspects of this disclosure, for example. One possible communication between a client 1002 and a server 1004 can be in the form of a data packet transmitted between two or more computer processes wherein the data packet may include video data. The data packet can include a metadata, e.g., associated contextual information, for example. The system 1000 includes a communication framework 1006 (e.g., a global communication network such as the Internet, or mobile network(s)) that can be employed to facilitate communications between the client(s) 1002 and the server(s) 1004.

Communications can be facilitated via a wired (including optical fiber) and/or wireless technology. The client(s) 1002 include or are operatively connected to one or more client data store(s) 1008 that can be employed to store information local to the client(s) 1002 (e.g., associated contextual information). Similarly, the server(s) 1004 are operatively include or are operatively connected to one or more server data store(s) 1010 that can be employed to store information local to the servers 1004. In one embodiment, a client 1002 can transfer an encoded file, in accordance with the disclosed subject matter, to server 1004. Server 1004 can store the file, decode the file, or transmit the file to another client 1002. It is to be appreciated, that a client 1002 can also transfer uncompressed file to a server 1004 and server 1004 can compress the file in accordance with the disclosed subject matter. Likewise, server 1004 can encode video information and transmit the information via communication framework 1006 to one or more clients 1002.

The present disclosure may be a system, a method, an apparatus and/or a computer program product at any possible technical detail level of integration. The computer program product can include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system comprising:
a memory that stores computer executable components;
a processor that executes the computer executable components stored in the memory, wherein the computer executable components comprise:
a transmission component configured to retrieve a set of genetic data from one or more device capable of analyzing genetic material;
an identification component configured to identify a first subset of genetic data representing a star allele that corresponds to a set of phenotypic traits;
a first generation component configured to generate a set of output data based on correlations between the first subset of genetic data, clinical data and guidance data;
a scoring component that assigns a score to respective subsets of output data based on a set of scoring requirements; and
a first determination component that determines a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

2. A system comprising:
a memory that stores computer executable components;
a processor that executes the computer executable components stored in the memory, wherein the computer executable components comprise:
a second generation component configured to generate a set of pharmacogenetics data based on a coupling of a set of identification data to a set of client data; and
a summarization component configured to summarize the set of pharmacogenetics data for presentation at a user interface.

3. A system comprising:
a memory that stores computer executable components;
a processor that executes the computer executable components stored in the memory, wherein the computer executable components comprise:
a third generation component configured to generate assay data corresponding to a group of biomarkers representing pharmacogenetic factors that indicate addiction susceptibility;
a second determination component configured to determine a risk score based on the generated assay data based on a set of weighting factors; and
a prediction component configured to predict a likelihood of addiction based on the risk score.

4. A system comprising:
a memory that stores computer executable components;
a processor that executes the computer executable components stored in the memory, wherein the computer executable components comprise:
an analysis component configured to evaluate a set of employer expenditure data;
a matching component configured to match the employer expenditure data to a set of pharmacogenetic data; and
an impact analysis component configured to determine an impact of pharmacogenetic treatment data on the employer expenditure data.

5. A computer-implemented method, comprising:
retrieving, by a system operatively coupled to a processor, a set of genetic data from one or more device capable of analyzing genetic material;
identifying, by the system, a first subset of genetic data representing a star allele that corresponds to a set of phenotypic traits;
generate, by the system, a set of output data based on correlations between the first subset of genetic data, clinical data and guidance data;
assigning, by the system, a score to respective subsets of output data based on a set of scoring requirements; and
determining, by the system, a target subset of output data of the subsets of output data to present at a user interface of a device based on the target subset of output data being greater than a threshold score, and wherein the target subset of output data represents information corresponding to an absorption, metabolization, or elimination reaction of a medication in association with the first subset of genetic data.

6. A computer-implemented method, comprising:
generating, by a system operatively coupled to a processor a set of pharmacogenetics data based on a coupling of a set of identification data to a set of client data; and
summarizing, by the system, the set of pharmacogenetics data for presentation at a user interface.

7. A computer-implemented method, comprising:
generating, by a system operatively coupled to a processor, assay data corresponding to a group of biomarkers representing pharmacogenetic factors that indicate addiction susceptibility;
determining, by the system, a risk score based on the generated assay data based on a set of weighting factors; and
predicting, by the system, a likelihood of addiction based on the risk score.

8. A computer-implemented method, comprising:
evaluating, by a system operatively coupled to a processor, a set of employer expenditure data;
matching, by the system, the employer expenditure data to a set of pharmacogenetic data; and
determining, by the system, an impact of pharmacogenetic treatment data on the employer expenditure data.
